SEQUENCE LISTING (20 sequences)

SEQ ID NO: 1 - DNA, 1023 bp, Homo sapiens
atggagccgggcaggacccagataaagcttgaccccaggtacacagcagatcttctggaggtgctgaagaccaattacggcatcccctccgcctgcttctctcagcctcccacagcagcccaactcctgagagccctgggccctgtggaacttgccctcactagcatcctgaccttgctggcgctgggctccattgccatcttcctggaggatgccgtctacctgtacaagaacaccctttgccccatcaagaggcggactctgctctggaagagctcggcacccacggtggtgtctgtgctgtgctgctttggtctctggatccctcgttccctggtgctggtggaaatgaccatcacctcgttttatgccgtgtgcttttacctgctgatgctggtcatggtggaaggctttggggggaaggaggcagtgctgaggacgctgagggacaccccgatgatggtccacacaggcccctgctgctgctgctgcccctgctgtccacggctgctgctcaccaggaagaagcttcagctgctgatgttgggccctttccaatacgccttcttgaagataacgctgaccctggtgggcctgtttctcgtccccgacggcatctatgacccagcagacatttctgaggggagcacagctctatggatcaacactttccttggcgtgtccacactgctggctctctggaccctgggcatcatttcccgtcaagccaggctacacctgggtgagcagaacatgggagccaaatttgctctgttccaggttctcctcatcctgactgccctacagccctccatcttctcagtcttggccaacggtgggcagattgcttgttcgcctccctattcctctaaaaccaggtctcaagtgatgaattgccacctcctcatactggagacttttctaatgactgtgctgacacgaatgtactaccgaaggaaagaccacaaggttgggtatgaaactttctcttctccagacctggacttgaacctcaaagcctaa

SEQ ID NO: 2 - protein, 340 aa, Homo sapiens
MEPGRTQIKLDPRYTADLLEVLKTNYGIPSACFSQPPTAAQLLRALGPVELALTSILTLLALGSIAIFLEDAVYLYKNTLCPIKRRTLLWKSSAPTVVSVLCCFGLWIPRSLVLVEMTITSFYAVCFYLLMLVMVEGFGGKEAVLRTLRDTPMMVHTGPCCCCCPCCPRLLLTRKKLQLLMLGPFQYAFLKITLTLVGLFLVPDGIYDPADISEGSTALWINTFLGVSTLLALWTLGIISRQARLHLGEQNMGAKFALFQVLLILTALQPSIFSVLANGGQIACSPPYSSKTRSQVMNCHLLILETFLMTVLTRMYYRRKDHKVGYETFSSPDLDLNLKA

SEQ ID NO: 3 - DNA, 1122 bp, Homo sapiens
atggagcagcctgtgttcctgatgacaactgccgctcaggccatctctggcttcttcgtgtggacggccctgctcatcacatgccaccagatctacatgcacctgcgctgctacagctgccccaacgagcagcgctacatcgtgcgcatcctcttcatcgtgcccatctacgcctttgactcctggctcagcctcctcttcttcaccaacgaccagtactacgtgtacttcggcaccgtccgcgactgctatgaggccttggtcatctataatttcctgagcctgtgctatgagtacctaggaggagaaagttccatcatgtcggagatcagaggaaaacccattgagtccagctgtatgtatggcacctgctgcctctggggaaagacttattccatcggatttctgaggttctgcaaacaggccaccctgcagttctgtgtggtgaagccactcatggcggtcagcactgtggtcctccaggccttcggcaagtaccgggatggggactttgacgtcaccagtggctacctctacgtgaccatcatctacaacatctccgtcagcctggccctctacgccctcttcctcttctacttcgccacccgggagctgctcagcccctacagccccgtcctcaagttcttcatggtcaagtccgtcatctttctttccttctggcaaggcatgctcctggccatcctggagaagtgtggggccatccccaaaatccactcggcccgcgtgtcggtgggcgagggcaccgtggctgccggctaccaggacttcatcatctgtgtggagatgttctttgcagccctggccctgcggcacgccttcacctacaaggtctatgctgacaagaggctggacgcacaaggccgctgtgcccccatgaagagcatctccagcagcctcaaggagaccatgaacccgcacgacatcgtgcaggacgccatccacaacttctcacctgcctaccagcagtacacgcagcagtccaccctggagcctgggcccacctggcgtggtggcgcccacggcctctcccgctcccacagcctcagtggcgcccgcgacaacgagaagactctcctgctcagctctgatgatgaattctag

SEQ ID NO: 4 - protein, 373 aa, Homo sapiens
MEQPVFLMTTAAQAISGFFVWTALLITCHQIYMHLRCYSCPNEQRYIVRILFIVPIYAFDSWLSLLFFTNDQYYVYFGTVRDCYEALVIYNFLSLCYEYLGGESSIMSEIRGKPIESSCMYGTCCLWGKTYSIGFLRFCKQATLQFCVVKPLMAVSTVVLQAFGKYRDGDFDVTSGYLYVTIIYNISVSLALYALFLFYFATRELLSPYSPVLKFFMVKSVIFLSFWQGMLLAILEKCGAIPKIHSARVSVGEGTVAAGYQDFIICVEMFFAALALRHAFTYKVYADKRLDAQGRCAPMKSISSSLKETMNPHDIVQDAIHNFSPAYQQYTQQSTLEPGPTWRGGAHGLSRSHSLSGARDNEKTLLLSSDDEF

SEQ ID NO: 5 - DNA, 1242 bp, Homo sapiens
atgagtaatgtctcagggatcctggagacagccggcgtccccctggtgtcagcgaactggccgcagcccagccccccaccggctgtgccagctgggccgcagatggaccacatggggaacagctcccagggggccccctggctcttcctcacctccgcactggcccgaggcgtctcggggatcttcgtgtggactgccctggtgctcacctgccaccagatctatctgcacctgcgctcctacaccgtgccacaggagcaacgttacatcatccgcctgctcctcatcgtgcccatctacgccttcgactcctggctcagcctcctcctcctcggagaccaccagtactacgtctacttcgactctgtgcgggactgctacgaagcctttgtcatttacagcttcctgagcctgtgtttccagtacctgggaggcgagggcgccatcatggctgagattcgtggaaagcccatcaagtccagctgcttgtacggcacctgctgcctccggggcatgacctactccatcgggttcctgcgcttctgtaagcaggccactctgcagttctgcctggtgaagcccgtcatggccgtcaccaccatcatcctccaggcatttggcaaataccacgacggggacttcaatgtccgcagcggctacctctatgtgaccctcatctacaacgcctccgtcagcctcgccctctacgccctgttcctcttctacttcaccaccagggagctcctgcggcccttccagcccgtcctcaagttcctcaccatcaaagccgtcatcttcctgtcgttctggcaagggctgctgctggccatcctggagcggtgcggggtcatcccggaggtggagaccagcggcgggaacaagctgggggctggcacgctggccgccggctaccagaacttcatcatctgcgtggagatgctgttcgcctccgtggccctgcgttatgccttcccctgccaggtgtacgcagagaagaaggagaattcaccagcccccccggcacccatgcagagcatctccagcggcatcagggagacagtgagcccccaggacatcgtgcaggacgccatccacaacttctcccccgcctaccagcactacacgcagcaggccacgcacgaggcgcccaggcccggcacccaccccagcggcggctccggcgggagcaggaagagccggagcctggagaagcggatgctgatcccctcggaggacctgtag

SEQ ID NO: 6 - protein, 413 aa, Homo sapiens
MSNVSGILETAGVPLVSANWPQPSPPPAVPAGPQMDHMGNSSQGAPWLFLTSALARGVSGIFVWTALVLTCHQIYLHLRSYTVPQEQRYIIRLLLIVPIYAFDSWLSLLLLGDHQYYVYFDSVRDCYEAFVIYSFLSLCFQYLGGEGAIMAEIRGKPIKSSCLYGTCCLRGMTYSIGFLRFCKQATLQFCLVKPVMAVTTIILQAFGKYHDGDFNVRSGYLYVTLIYNASVSLALYALFLFYFTTRELLRPFQPVLKFLTIKAVIFLSFWQGLLLAILERCGVIPEVETSGGNKLGAGTLAAGYQNFIICVEMLFASVALRYAFPCQVYAEKKENSPAPPAPMQSISSGIRETVSPQDIVQDAIHNFSPAYQHYTQQATHEAPRPGTHPSGGSGGSRKSRSLEKRMLIPSEDL

SEQ ID NO: 7 - DNA, 1317 bp, Homo sapiens
atgccttgcacttgtacctggaggaactggagacagtggattcgacctttagtagcggtcatctacctggtgtcaatagtggttgcggttcccctatgcgtgtgggaattacagaaactggaggttggaatacacaccaaggcttggtttattgctggaatctttttgctgttgactattcctatatcactgtgggtgatattgcaacacttagtgcattatacacaacctgaactacaaaaaccaataataaggattctttggatggtacctatttacagtttagatagttggatagctttgaaatatcccggaattgcaatatatgtggatacctgcagagaatgctatgaagcttatgtaatttacaactttatgggattccttaccaattatctaactaaccggtatccaaatctggtattaatccttgaagccaaagatcaacagaaacatttccctcctttatgttgctgtccaccatgggctatgggagaagtattgctgtttaggtgcaaactaggtgtattacagtacacagttgtcagacctttcaccaccatcgttgctttaatctgtgagctgcttggtatatatgacgaagggaactttagcttttcaaatgcttggacttatttggttataataaacaacatgtcacagttgtttgccatgtattgtctcctgctcttttataaagtactaaaagaagaactgagcccaatccaacctgttggcaaatttctttgtgtaaggctggtggtttttgtttctttttggcaagcagtagttattgctttgttggtaaaagttggcgttatttctgaaaagcatacgtgggaatggcaaactgtagaagctgtggccaccggactccaggattttattatctgtattgagatgttcctcgctgccattgctcatcattacacattctcatataaaccatatgtccaagaagcagaagagggctcatgctttgattcctttcttgccatgtgggatgtctcagatattagagatgatatttctgaacaagtaaggcatgttggacggacagtcgggggacatcccaggaaaaaattgtttcccgaggatcaagatcaaaatgaacatacaagtttattatcatcatcatcacaagatgcaatttccattgcttcttctatgccaccttcacccatgggtcactaccaagggtttggacacactgtgactccccagactacacctaccacagctaagatatctgatgaaatccttagtgatactataggagagaaaaaagaaccttcagataaatccgtggattcctga

SEQ ID NO: 8 - protein, 438 aa, Homo sapiens
MPCTCTWRNWRQWIRPLVAVIYLVSIVVAVPLCVWELQKLEVGIHTKAWFIAGIFLLLTIPISLWVILQHLVHYTQPELQKPIIRILWMVPIYSLDSWIALKYPGIAIYVDTCRECYEAYVIYNFMGFLTNYLTNRYPNLVLILEAKDQQKHFPPLCCCPPWAMGEVLLFRCKLGVLQYTVVRPFTTIVALICELLGIYDEGNFSFSNAWTYLVIINNMSQLFAMYCLLLFYKVLKEELSPIQPVGKFLCVRLVVFVSFWQAVVIALLVKVGVISEKHTWEWQTVEAVATGLQDFIICIEMFLAAIAHHYTFSYKPYVQEAEEGSCFDSFLAMWDVSDIRDDISEQVRHVGRTVGGHPRKKLFPEDQDQNEHTSLLSSSSQDAISIASSMPPSPMGHYQGFGHTVTPQTTPTTAKISDEILSDTIGEKKEPSDKSVDS

SEQ ID NO: 9 - DNA, 24 bp, Artificial Sequence
  hOST 1 PCR primer 1
atggagcagcctgtgttcctgatg

SEQ ID NO: 10 - DNA, 25 bp, Artificial Sequence
  hOST 1 PCR primer 2
ctagaattcatcatcagagctgagc

SEQ ID NO: 11 - DNA, 25 bp, Artificial Sequence
  hOST 2 PCR primer 1
atgagtaatgtctcagggatcctgg

SEQ ID NO: 12 - DNA, 25 bp, Artificial Sequence
  hOST 2  PCR primer 2
ctacaggtcctccgaggggatcagc

SEQ ID NO: 13 - DNA, 24 bp, Artificial Sequence
  hOST 3 PCR primer 1
atgccttgcacttgtacctggagg

SEQ ID NO: 14 - DNA, 26 bp, Artificial Sequence
  hOST 3 PCR primer 2
tcaggaatccacggatttatctgaag

SEQ ID NO: 15 - DNA, 25 bp, Artificial Sequence
  hOST 4 PCR primer 1
atggagccgggcaggacccagataa

SEQ ID NO: 16 - DNA, 25 bp, Artificial Sequence
  hOST 4 PCR primer 2
ttaggctttgaggttcaagtccagg

SEQ ID NO: 17 - DNA, 1228 bp, Raja erinacea
cggacactcacagcgctggtcttaaccgcgcctgggatcccgacactgccaagaagatggatgtagctcaccctgaggaagtgaccaggttttctccagatatcttgatggaaaagttcaacgtttctgaggcgtgcttcctgccccctccgatatccatccaactcatactgcagctgacgtggttagacattggtgtctttgccgcattgaccgcgatgactgtgctcaccatcgccatttacctggagatcgtctgctacctgatggacaaggtgaagtgtcccatcaagagaaagactttgatgtggaacagtgcagctccaaccgtcatcgccatcacttcctgccttggtctctgggtcccacgagccatcatgttcgtggacatggcggctgccatgtactttggtgttggcttctacctgatgctgctgatcatcgtacaggggtacggtggagaggaggccatgctccaacacctggccacacacaccatccgtatcagcaccgggccctgctgctgctgctgcccctgtctaccccacatacacctcacacggcagaaatacaagatctttgtgctgggagctttccaagtggctttcctccggcctgccctcttcttgctgggcgtggtcttgtggacaaacggcctctatgacccagatgattggtcctccactagcatcttcctctggctgaacctgttcctgggcgtttccaccatcctggggctgtggccggtcaacgtcctcttccgacactccaaggtgctcatggccgaccagaagctgacctgcaagtttgctctgttccaggctatcctgatcctgtcctcgctacagaattccatcattggaacgctggcgggagcggggcacattggctgtgctcctccctattctgcaaggaccagaggacagcaaatgaacaaccagctgttgattatcgagatgttcttcgttggtatcctgacgcggatttcctacaggaagagggatgaccgaccgggacaccgacatgtcggtgaggtccagcagattgtcagagaatgtgatcaaccagccatcgccgaccaacaggctgatcactccagcatctcccacatataaacacagatcagcaacataactacttggattaaaatgtgagattttgcatggagaaaaaaaaaaaaaaaaaaaaaaaaaaaaaaaaaaaaaaaaaaaaaaaaaaaaaaaaaaaa

SEQ ID NO: 18 - protein, 352 aa, Raja erinacea
MDVAHPEEVTRFSPDILMEKFNVSEACFLPPPISIQLILQLTWLDIGVFAALTAMTVLTIAIYLEIVCYLMDKVKCPIKRKTLMWNSAAPTVIAITSCLGLWVPRAIMFVDMAAAMYFGVGFYLMLLIIVQGYGGEEAMLQHLATHTIRISTGPCCCCCPCLPHIHLTRQKYKIFVLGAFQVAFLRPALFLLGVVLWTNGLYDPDDWSSTSIFLWLNLFLGVSTILGLWPVNVLFRHSKVLMADQKLTCKFALFQAILILSSLQNSIIGTLAGAGHIGCAPPYSARTRGQQMNNQLLIIEMFFVGILTRISYRKRDDRPGHRHVGEVQQIVRECDQPAIADQQADHSSISHI

SEQ ID NO: 19 - DNA, 454 bp, Homo sapiens
ctcgttgcacacgctaccaggagcaggggcatggagcacagtgagggggctcccggagacccagccggtactgtggtaccccaggagctgctggaagagatgctttggttttttcgtgtggaagatgcatctccctggaatcattccatccttgccctggcagctgtggtggtcattataagcatggtcctcctgggaagaagcatccaggcaagcagaaaagaaacgatgcagccaccagaaaaagaaactccagaagtcctgcatttggatgaggccaaggatcacaacagcctaaacaacctaagagaaactttgctctcagaaaagccaaacttggcccaggtggaacttgagttaaaagagagagatgtgctgtcagttttccttccggatgtaccagaaactgagagctagtgagggttcagagaagccccatcctaagccagacaca

SEQ ID NO: 20 - protein, 128 aa, Homo sapiens
MEHSEGAPGDPAGTVVPQELLEEMLWFFRVEDASPWNHSILALAAVVVIISMVLLGRSIQASRKETMQPPEKETPEVLHLDEAKDHNSLNNLRETLLSEKPNLAQVELELKERDVLSVFLPDVPETES

(19) United States
(12) Patent Application Publication
(10) Pub. No.: US 2004/0161424 A1
(43) Pub. Date: Aug. 19, 2004

(54) Human organic solute transporters

(75) Inventors: Noa Zerangue, Sunnyvale, CA (US); Christopher J. Paddon, Pacifica, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: XenoPort, Santa Clara, CA

(21) Appl. No.: 10/763,644

(22) Filed: Jan. 22, 2004

Related U.S. Application Data
(63) Continuation-in-part of application No. PCT/US03/32087, filed on Oct. 8, 2003.
(60) Provisional application No. 60/417,298, filed on Oct. 8, 2002.

(51) Int. Cl. C12Q 1/68; C07H 21/04; C07K 14/705; A61K 39/395

(52) U.S. Cl. 424/143.1; 435/6; 435/69.1; 435/320.1; 435/325; 530/350; 530/388.22; 536/23.5

(57) ABSTRACT

The invention provides several new transporter proteins, nucleic acids encoding them and methods of using the transporters to screen agents, conjugates or conjugate moieties, linked or linkable to agents, for capacity to be transported as substrates through the transporters. The invention also provides methods of treatment involving oral delivery of agents that either alone, or as a result of linkage to a conjugate moiety, are substrates of one of the transporter.

### CROSS-REFERENCE TO RELATED APPLICATION

This application is a continuation-in-part of PCT/US03/32087, filed on Oct. 8, 2003, written in English and designating the United States, which is a nonprovisional and claims the benefit under 35 USC 119(e) of U.S. Ser. No. 60/417,298 filed Oct. 8, 2002, both of which are incorporated by reference in their entirety for all purposes.

### BACKGROUND OF THE INVENTION

Recent advances in the pharmaceutical industry have resulted in the formation of an increasing number of potential therapeutic agents. However, formulating the compounds for effective oral bioavailability has proven difficult because of problems associated with uptake and high susceptibility to metabolic enzymes. Natural transporter proteins are involved in the uptake of various molecules into and/or through cells. One strategy for the delivery is to identify pharmacological agents that are, or can be modified to be, substrates for transport proteins.

In general, two major transport systems exist: solute carrier-mediated systems and receptor mediated systems. Carrier-mediated systems use transport proteins that are anchored to the cell membrane, typically by a plurality of membrane-spanning loops and function by transporting their substrates via an energy-dependent flip-flop or other mechanism, exchange and other facilitative or equilibrative mechanisms. Carrier-mediated transport systems are involved in the active or non-active, facilitated transport of many important nutrients such as vitamins, sugars, and amino acids, as well as xenobiotic compounds. The carrier systems result in result in transport into the enterocytes from blood or lumen, and across the epithelial cell layer from lumen into blood (absorption) or blood to lumen (secretion). Carrier-mediated transporters are also present in organs such as liver and kidney, in which the proteins are involved in the excretion or re-absorption of circulating compounds.

Receptor-mediated transport systems differ from the carrier-mediated systems in that receptors usually span the cell membrane only a single time. Furthermore, substrate binding triggers an invagination and encapsulation process that results in the formation of various transport vesicles to carry the substrate (and sometimes other molecules) into and through the cell. This process of membrane deformations that result in the internalization of certain substrates and their subsequent targeting to certain locations in the cytoplasm is generally referred to as endocytosis.

Polar or hydrophilic compounds are typically poorly absorbed through an animal's intestine as there is a substantial energetic penalty for passage of such compounds across the lipid bilayers that constitute cellular membranes. Many nutrients that result from the digestion of ingested foodstuffs in animals, such as amino acids, di- and tripeptides, monosaccharides, nucleosides and water-soluble vitamins, are polar compounds whose uptake is essential to the viability of the animal. For these substances there exist specific mechanisms for active transport of the solute molecules across the apical membrane of the intestinal epithelia. This transport is frequently energized by co-transport of ions down a concentration gradient.

An organic solvent transporter and an ancillary membrane protein, termed Ostα and β, have recently been cloned from skate, Wang et al., PNAS 98, 9431-9436 (2001). Ostα encodes a protein of 352 amino acids and seven putative transmembrane domains. Ostβ encodes a protein of 182 amino acids with at least one and perhaps two transmembrane domains. Xenopus oocytes transfected with nucleic acids encoding Ostα and β were reported to transport labeled taurocholate. Wang et al., report searching databases for sequences showing significant sequence identity with Ostα and β but did not find any such sequences.

### SUMMARY OF THE INVENTION

The present invention provides new transporter polypeptides in isolated form. Isolated polypeptides of the invention are at least 80% identical to an amino acid sequence as set forth in a sequence selected from the group consisting of SEQ ID NOS:2, 4, 6, and 8, over a region of at least 40 amino acids in length when compared using the BLASTP algorithm with a wordlength (W) of 3, and the BLOSUM62 scoring matrix. Some polypeptides of the invention specifically binds to an antibody that specifically binds to a polypeptide selected from the group consisting of SEQ ID NOS: 2, 4, 6, and 8. SEQ ID NOS: 2, 4, 6 and 8 are exemplary amino acid sequences of transporter proteins.

The invention also provides isolated nucleic acids having a sequence at least 80% identical to a polynucleotide having a sequence selected from the group consisting of SEQ ID NOS: 1, 3, 5, and 7 over a region at least 100 nucleotides in length when compared using the BLASTN algorithm with a wordlength (W) of 11, M=5, and N=−4. Some isolated nucleic acids of the invention hybridize to a sequence selected from the group consisting of SEQ ID NOS:1, 3, 5, and 7 under high stringency conditions, including 50% formamide, 5×SSC, 5×Denhardt's solution, 10 mM sodium phosphate, pH 6.5, 100 mg/ml salmon sperm DNA at 42°. SEQ ID NOS: 1, 3, 5, and 7 are exemplary nucleic acids of the invention. Optionally, the isolated nucleic acids are provided as components of a vector.

The invention further provides screening methods to determine whether an agent, conjugate or conjugate moiety is a substrate of a transporter. Some methods include providing a cell expressing a nucleic acid as described above in the outer membrane of the cell, contacting the cell with an agent, conjugate moiety or conjugate, and determining whether the agent, conjugate moiety or conjugate passes through the transporter. In some methods, the transporter encoded by a nucleic acid has the sequence of SEQ ID NO:2. In some methods, the cells used for expression of the transporter are Chinese hamster ovary cells, human embryonic kidney cells or oocytes.

The invention further provides methods to determine whether an agent, conjugate moiety or conjugate binds a transporter. The transporter, which has a sequence with at least 80% sequence identity to an amino acid sequence as set forth in a sequence selected from the group consisting of SEQ ID NOS:2, 4, 6, and 8, over a region of at least 40 amino acids in length when compared using the BLASTP algorithm with a wordlength (W) of 3, and the BLOSUM62 scoring matrix, is contacted with an agent, conjugate moiety or conjugate and the presence or absence of binding between the transporter and the agent, conjugate moiety or conjugate is detected.

The invention further provides a conjugate comprising an agent linked to a conjugate moiety for a transporter polypeptide as described above. The conjugate shows a Vmax of at least 1% of taurocholate for the transporter wherein the agent has a pharmaceutical activity without the conjugate moiety, and the conjugate has a greater Vmax for the transporter than the agent without the conjugate moiety.

The invention further provides methods of manufacturing a pharmaceutical composition, comprising linking an agent to a conjugate moiety to form a conjugate wherein the conjugate is transported by a transporter having an amino acid sequence with at least 80% identity to an amino acid sequence as set forth in a sequence selected from the group consisting of SEQ ID NOS:2, 4, 6, and 8, over a region of at least 40 amino acids in length when compared using the BLASTP algorithm with a wordlength (W) of 3, and the BLOSUM62 scoring matrix, with a Vmax higher than the agent alone, formulating the conjugate with a carrier as a pharmaceutical composition.

The invention further provides methods of treatment comprising administering to patient a conjugate comprising an agent linked to a conjugate moiety wherein the conjugate is transported by a transporter having an amino acid sequence with at least 80% identity to an amino acid sequence as set forth in a sequence selected from the group consisting of SEQ ID NOS:2, 4, 6, and 8, over a region of at least 40 amino acids in length when compared using the BLASTP algorithm with a wordlength (W) of 3, and the BLOSUM62 scoring matrix with a Vmax higher than the agent alone. In some methods the conjugate is administered orally to the patient. In other methods, the conjugate is administered intravenously.

### DEFINITIONS

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally-occurring nucleotides. Unless otherwise indicated, a particular nucleic acid sequence includes the complementary sequence thereof. A “subsequence” refers to a sequence of nucleotides or amino acids that comprise a part of a longer sequence of nucleotides or amino acids (e.g., a polypeptide), respectively.

A “probe” is a nucleic acid capable of binding to a target nucleic acid of complementary sequence through one or more types of chemical bonds, usually through complementary base pairing, usually through hydrogen bond formation, thus forming a duplex structure. The probe binds or hybridizes to a “probe binding site.” A probe may include natural (i.e., A, G, C, or T) or modified bases (7-deazaguanosine, inosine, etc.). A probe can be an oligonucleotide which is a single-stranded DNA. Oligonucleotide probes can be synthesized or produced from naturally occurring polynucleotides. In addition, the bases in a probe can be joined by a linkage other than a phosphodiester bond, so long as it does not interfere with hybridization. Thus, probes may be peptide nucleic acids in which the constituent bases are joined by peptide bonds rather than phosphodiester linkages (see, for example, Nielsen et al., Science 254, 1497-1500 (1991)). Some probes may have leading and/or trailing sequences of noncomplementary flanking a region of complementarity.

The terms “polypeptide,” “peptide” and “protein” are used interchangeably to refer to a polymer of amino acid residues. The term also applies to amino acid polymers in which one or more amino acids are chemical analogues of corresponding naturally-occurring amino acids.

The term “operably linked” refers to functional linkage between a nucleic acid expression control sequence (such as a promoter, signal sequence, or array of transcription factor binding sites) and a second polynucleotide, wherein the expression control sequence affects transcription and/or translation of the second polynucleotide.

A “heterologous sequence” or a “heterologous nucleic acid,” as used herein, is one that originates from a source foreign to the particular host cell, or, if from the same source, is modified from its original form. Thus, a heterologous gene in a prokaryotic host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “recombinant” when used with reference to a cell indicates that the cell replicates a heterologotis nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

The term “isolated,” “purified” or “substantially pure” means an object species that has been enriched or separated from the components in its native environment. Thus, a nucleic acid that is being recombinantly expressed in vitro is isolated notwithstanding that the nucleic acid is surrounded by other cellular components. The term may also indicate the an object species is the predominant macromolecular species present (i.e., on a molar basis it is more abundant than any other individual species in the composition), and preferably the object species comprises at least about 50 percent (on a molar basis) of all macromolecular species present. Generally, an isolated, purified or substantially pure composition will comprise more than 80 to 90 percent of all macromolecular species present in a composition. Most preferably, the object species is purified to essential homogeneity (i.e., contaminant species cannot be detected in the composition by conventional detection methods) wherein the composition consists essentially of a single macromolecular species.

The term “complementary” means that one nucleic acid is identical to, or hybridizes selectively to, another nucleic acid molecule. Selectivity of hybridization exists when hybridization occurs that is more selective than total lack of specificity. Typically, selective hybridization will occur when there is at least about 55% identity over a stretch of at least 14-25 nucleotides, preferably at least 65%, more preferably at least 75%, and most preferably at least 90%. Preferably, one nucleic acid hybridizes specifically to the other nucleic acid. See M. Kanehisa, Nucleic Acids Res. 12:203 (1984).

The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below for example, or by visual inspection.

The phrase “substantially identical,” in the context of two nucleic acids or polypeptides, refers to two or more sequences or subsequences that have at least 75%, preferably at least 85%, more preferably at least 90%, 95% or higher nucleotide or aminoacid residue identity, when compared using a sequence comparison algorithm such as those described below for example, or by visual inspection. Preferably, the substantial identity exists over a region of the sequences that is at least about 40-50 residues in length, preferably over a longer region than 50 amino acids, more preferably at least about 90-100 residues, and most preferably the sequences are substantially identical over the full length of the sequences being compared, such as the coding region of a nucleotide for example.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted,

e.g., by the local homology algorithm of Smith & Waterman, Adv. Appl. Math. 2:482, (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide.

A further indication that two nucleic acid sequences or polypeptides are substantially identical is that the polypeptide encoded by the first nucleic acid is immunologically cross reactive with the polypeptide encoded by the second nucleic acid, as described below. The phrases “specifically binds to a protein” or “specifically immunoreactive with,” when referring to an antibody refers to a binding reaction which is determinative of the presence of the protein in the presence of a heterogeneous population of proteins and other biologics. Thus, under designated immunoassay conditions, a specified antibody binds preferentially to a particular protein and does not bind in a significant amount to other proteins present in the sample. Specific binding to a protein under such conditions requires an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

A polypeptide is typically substantially identical to a second polypeptide, for example, where the two peptides differ only by conservative substitutions. A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well-known in the art. See, e.g., Creighton (1984) Proteins, W.H. Freeman and Company. In addition, individual substitutions, deletions or additions which alter, add or delete a single amino acid or a small percentage of amino acids in an encoded sequence are also “conservatively modified variations.”

Allelic variants of a gene refer to variant forms of the same gene between different individuals of the same species. Cognate forms of a gene refers to variation between structurally and functionally related genes between species. For example, the human gene showing the greatest sequence identity and functional related to a mouse gene is the human cognate form of the mouse gene.

The term “naturally-occurring“as applied to an object refers to the fact that an object can be found in nature. For example, a polypeptide or polynucleotide sequence that is present in an organism that can be isolated from a source in nature and which has not been intentionally modified by humans in the laboratory is naturally-occurring.

The term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “patient” includes human and veterinary subjects.

The phrases “specifically binds” when referring to a protein or “specifically immunoreactive with” when referring to an antibody, refers to a binding reaction which is determinative of the presence of the protein in the presence of a heterogeneous population of proteins and other biologics. Thus, under designated conditions, a specified ligand binds preferentially to a particular protein and does not bind to a significant extentto other proteins present in the sample. A molecule such as antibody that specifically binds to a protein often has an association constant of at least 10 M⁻¹, 10⁶ M⁻¹ or 10⁷ M⁻¹, preferably 10⁸ M⁻¹ to 10⁹ M⁻¹, and more preferably, about 10¹⁰ M⁻¹ to 10¹¹ M⁻¹ or higher. However, some substrates of a transporter have much lower affinities of the order of 10-10³ M⁻¹ and yet the binding can still be shown to be specific. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

A “transport protein” is a protein that has a direct or indirect role in transporting a molecule into and/or through a cell. The term includes, for example, membrane-bound proteins that recognize a substrate and effects its entry into, or exit from a cell by a carrier-mediated transporter or by receptor-mediated transport. These proteins are sometimes referred to as transporter proteins. The term also includes intracellularly expressed proteins that participate in trafficking of substrates through or out of a cell. The term also includes proteins or glycoproteins exposed on the surface of a cell that do not directly transport a substrate but bind to the substrate holding it in proximity to a receptor or transporter protein that effects entry of the substrate into or through the cell. Examples of carrier proteins include: the intestinal and liver bile acid transporters, dipeptide transporters, oligopeptide transporters, simple sugar transporters (e.g., SGLT1), phosphate transporters, monocarboxcylic acid transporters, P-glycoprotein transporters, organic anion transporters (OATP), and organic cation transporters. Examples of receptor-mediated transport proteins include: viral receptors, immunoglobulin receptors, bacterial toxin receptors, plant lectin receptors, bacterial adhesion receptors, vitamin transporters and cytokine growth factor receptors.

A “substrate” of a transport protein is a compound whose uptake into or passage through a cell is facilitated by the transport protein.

The term “ligand” of a transport protein includes substrates and other compounds that bind to the transport protein without being taken up or transported through a cell. Some ligands by binding to the transport protein inhibit or antagonize uptake of the substrate or passage of substrate through a cell by the transport protein. Some ligands by binding to the transport protein promote or agonize uptake or passage of the compound by the transport protein or another transport protein. For example, binding of a ligand to one transport protein can promote uptake of a substrate by a second transport protein in proximity with the first transport protein.

The term “agent” is used to describe a compound that has or may have a pharmacological activity. Agents include compounds that are known drugs, compounds for which pharmacological activity has been identified but which are undergoing further therapeutic evaluation, and compounds that are members of collections and libraries that are to be screened for a pharmacological activity.

An agent is “orally active” if it can exert a pharmaceutical activity when administered via an oral route.

A “conjugate moiety” refers to a compound or part of a compound that does not itself have pharmacological activity but which can be linked to an agent to form a conjugate that does have pharmacological activity. Typically, the agent has pharmacologic activity without the conjugate moiety. The conjugate moiety facilitates therapeutic use of the agent by promoting uptake of the agent via a transporter. A conjugate moiety can itself be a substrate for a transporter or can become a substrate when linked to a compound (e.g., valacyclovir). Thus, a conjugate moiety formed from a compound and a conjugate moiety can have higher uptake activity than either the compound or moiety alone.

A “pharmacological” activity means that an agent exhibits an activity in a screening system that indicates that the agent is or may be useful in the prophylaxis or treatment of a disease. The screening system can be in vitro, cellular, animal or human. Agents can be described as having pharmacological activity notwithstanding that further testing may be required to establish actual prophylactic or therapeutic utility in treatment of a disease.

Vmax and Km of a compound for a transporter are defined in accordance with convention. Vmax is the number of molecules of compound transported per second at saturating concentration of the compound. Km is the concentration of the compound at which the compound is transported at half of Vmax. In general, a high value of Vmax is desirable for a substrate of a transporter. A low value of Km is desirable for transport of low concentrations of a compound, and a high value of Km is desirable for transport of high concentrations of a compound. Vmax is affected both by the intrinsic turnover rate of a transporter (molecules/transporter protein) and transporter density in plasma membrane which depends on expression level. For these reasons, the intrinsic capacity of a compound to be transported by a particular transporter is usually expressed as the ratio Vmax of the compound/Vmax of a control compound known to be a substrate for the transporter.

### DETAILED DESCRIPTION OF THE INVENTION

I. Introduction

The invention provides several new transporter protein, nucleic acids encoding them and methods of using the transporters to screen agents, conjugates or conjugate moieties, linked or linkable to agents, for capacity to be transported as substrates through the transporters. The invention also provides methods of treatment involving oral delivery of agents that either alone, or as a result of linkage to a conjugate moiety, are substrates of one of the transporter.

II. The Transporter Proteins

The invention provides nucleic acid and amino acid sequences for several human organic solvent transporter proteins, designated hOst-1, hOst-2, hOst3 and hOst-4. One transporter protein, hOst-4, is believed to be the human cognate form of the skate Ostα gene. The nucleic acid and amino acid sequences of hOst-4 are designated SEQ. ID. NOS: 1 and 2. Ost4 shows about 38% sequence identity with the skate Ostα protein sequence (SEQ ID NO:17)) (see Table 1).

TABLE 1
Pairwise Percent Identity of Peptide Sequences

| | | | | | skate |
|---|---|---|---|---|---|
| | hOST1 | hOST2 | hOST3 | hOST4 | OSTα |
| hOST1 | 100% | 64% | 34% | 17% | 18% |
| hOST2 | 64% | 100% | 33% | 17% | 19% |
| hOST3 | 34% | 33% | 100% | 19% | 18% |
| hOST4 | 17% | 17% | 19% | 100% | 38% |
| skateOSTα | 18% | 19% | 18% | 38% | 100% |

The theoretical location of transmembrane domains is shown in Table 2 below. The numbers refer to the location of amino acid residues, with the initiator methionine as the first amino acid. Transmembrane domains were allocated by alignment of all OST sequences with skate OSTα using ClustalW, and comparison to the transmembrane domains predicted in OST alpha by a Hidden Markov Model (Sonnhammer, et al. A Hidden Markov Model for Predicting Transmembrane Helices in Protein Sequences, Proceedings of the Sixth International Conference on Intelligent Systems for Molecular Biology, pages 175-182, Menlo Park, Calif., 1998. AAAI Press.). The predicted N-terminus is extracellular, while the C-terminus is intracellular.

Other structural features of OST genes include potential glycosylation sites, such as at amino acid residues 3-5 of hOST2 and at amino acid residues 203-205 of hOST3. In addition the OST genes contain cysteine residues in the fourth hydrophilic domain. The skate OSTα has a cluster of six cysteine residues between the fourth and fifth transmembrane domains. Likewise, hOST 1 and hOST2 have 2 cysteine residues, while hOST3 has 3 cysteines and hOST4 has 7 cysteine residues.

TABLE 2
Putative Transmembrane Location

| Gene | 1 | 2 | 3 | 4 | 5 | 6 | 7 |
|---|---|---|---|---|---|---|---|
| hOST1 | 12-34 | 47-67 | 80-101 | 139-159 | 173-195 | 215-237 | 263-280 |
| hOST2 | 57-77 | 90-109 | 123-144 | 182-201 | 216-238 | 158-280 | 307-324 |
| hOST3 | 48-71 | 83-103 | 113-134 | 172-191 | 206-228 | 248-270 | 295-312 |
| hOST4 | 53-75 | 88-108 | 118-139 | 177-200 | 214-237 | 256-278 | 300-317 |

Nucleic acid and amino acid sequences for hOst-1, hOst-2, and hOst3 are designated SEQ. ID. NOS: 3-8 respectively. The percentage sequence identities between the amino acid sequences are shown in Table 1. hOst-1, hOst-2 and hOst-3 are probably nonallelic with each other and hOst-4 but show significant structural and functional relatedness. The relationship is strongest between hOst-I and hOst-2, which show 64% sequence identity with each other. The amino acid sequence of a cotransporter that can be expressed in combination with hOst-1, hOst-2, hOst-3, and particularly, hOst-4 is provided as SEQ ID NO: 19.

hOst-4 was identified by searching sequence databases for sequences having similarity with skate Ostα. A sequence for hOst-4 was compiled from more than one contig. This sequence was then used to design primers to amplify hOst-4 cDNA. hOst-1, hOst-2 and hOst-3 were also identified by searching databases for sequence similarity with skate Ostα and compiling different parts of the coding regions. The nucleic acid and deduced amino acid sequences provided come directly from the compilation of sequences from the database search without resequencing of cDNAs as was done for hOst-4. Reference to one of the above transporter proteins includes the full-length molecule and other polypeptides having a similar activity. The term also includes mature forms of transporters lacking a signal sequence. The invention also provides variants of exemplified transporter proteins having an amino acid sequence at least 80% identical to an amino acid sequence as set forth in SEQ ID NOS: 2, 4, 6, or 8. More preferably, the variants are at least 85% identical, still more preferably at least 90% or 95% identical to the amino acid sequence of SEQ ID NOS: 2, 4, 6, or 8. The region of similarity between a variant and an exemplary SEQ. ID typically extends over a region of at least 40 amino acids in length, more preferably over a longer region than 40 amino acids such as 50, 60, 70 or 80 amino acids, and most preferably over the full length of the polypeptide. One example of an algorithm that is useful for comparing a polypeptide to a SEQ ID NO. is the BLASTP algorithm; suitable parameters include a word length (W) of 3, and a BLOSUM62 scoring matrix. Variants include allelic variants, splice variants and cognate variants from mammalian species, particularly primates, bovines, canines, felines and rodents.

Besides substantially full-length polypeptides, the present invention provides for biologically active fragments of the polypeptides. Biological activity may include transport of a substrate that is also transported by the full length protein. Other examples of significant biological activity include antibody binding (e.g., a fragment competes with a full-length sequence as set forth in SEQ ID NOS: 2, 4, 6, or 8) and immunogenicity (i.e., possession of epitopes that stimulate B- or T-cell responses against the fragment. Fragments ordinarily comprise at least 5 contiguous amino acids, typically at least 6 or 7 contiguous amino acids, more typically 8 or 9 contiguous amino acids, usually at least 10, 11 or 12 contiguous amino acids, preferably at least 13 or 14 contiguous amino acids, more preferably at least 16 contiguous amino acids, and most preferably at least 20, 40, 60 or 80 contiguous amino acids. Other examples of subsequences provided by the invention are amino acid sequences wherein 1 to 10 amino acids are removed from the N-terminal of C-terminal end of SEQ ID NOS: 2, 4, 6, or 8.

Transporter proteins of the invention often share at least one antigenic determinant in common with the amino acid sequence set forth in SEQ ID NOS: 2, 4, 6, or 8. The existence of such a common determinant is evidenced by cross-reactivity of the variant protein with any antibody prepared against the full-length polypeptide. Cross-reactivity may be tested using polyclonal sera against the full-length, but can also be tested using one or more monoclonal antibodies against the full-length protein.

Some transporter proteins of the invention have a modified polypeptide backbone. Illustrative examples of such modifications include chemical derivatizations of polypeptides, such as acetylations and carboxylations. Modifications also include glycosylation modifications and processing variants of a typical polypeptide. Such processing steps specifically include enzymatic modifications, such as ubiquitinization and phosphorylation. See, e.g., Hershko & Ciechanover, Ann. Rev. Biochem. 51:335-364 (1982). Modifications also include substitutions with nonnaturally occurring amino acids, such as D amino acids.

Expression date for the transporters in a panel of human tissues is provided in Table 3. It can be seen that hOst-1, -2, -3 and -4 are all expressed significantly in the human intestine.

TABLE 3
mRNA Abundance, transcripts/reaction, normalized to GAPDH

Human Intestine Mucosal Biopsy Samples

| | | DU- | | | A. | T. | S. | | | | | |
|---|---|---|---|---|---|---|---|---|---|---|---|---|
| | STOMACH | ODENUM | ILEUM | CECUM | COLON | COLON | COLON | RECTUM | KIDNEY | LIVER | OVARY | TESTIS |
| OST- | 15218 | 16789 | 12214 | 9644 | 7730 | 9050 | 10163 | 7957 | 18188 | 9743 | 12498 | 2367 |
| 1 | | | | | | | | | | | | |
| OST- | 10033 | 17643 | 15518 | 6728 | 10112 | 11130 | 10712 | 6604 | 1600 | 4987 | 0 | 750 |
| 2 | | | | | | | | | | | | |
| OST- | 1530 | 6970 | 9449 | 8378 | 8881 | 9471 | 10060 | 1639 | 1654 | 2993 | N.D. | N.D. |
| 3 | | | | | | | | | | | | |
| OST- | 4866 | 42267 | 65347 | 24009 | 51725 | 55623 | 19137 | 1242 | 11058 | 55863 | 9937 | 24683 |
| 4 | | | | | | | | | | | | |
| (OST- | | | | | | | | | | | | |
| alpha) | | | | | | | | | | | | |

III. Nucleic Acids

The present invention further provides isolated and/or recombinant nucleic acids that encode the transporters of the invention or fragments thereof discussed above. The nucleic acids of the invention include naturally occurring, synthetic, and intentionally manipulated polynucleotide sequences (e.g., site directed mutagenesis or use of alternate promoters for RNA transcription). The nucleic acids of the invention also include sequences that are degenerate as a result of the degeneracy of the genetic code.

The polynucleotide encoding transporters with SEQ ID NOS: 2, 4, 6, or 8 are SEQ ID NOS: 1, 3, 5, and 7 respectively. Also included in the invention are subsequences of the above-described nucleic acid sequences. Such subsequences include, for example, the coding region of SEQ ID NOS: 1, 3, 5, and 7, with or without signal sequences, as well as subsequences that are at least 17, 18, 19, 20, 21, 22, 23, 24 or 25 nucleotides in length.

The invention also includes polynucleotide sequences that are typically substantially identical to a polynucleotide sequence of SEQ. ID NOS: 1, 3, 5, and 7. For example, the invention includes polynucleotide sequences that are at least about 80% identical to the nucleic acid SEQ ID NO: 1 over a region of at least about 100 nucleotides in length. More preferably, the nucleic acids of the invention are at least 85% identical to the nucleic acid sequence shown in SEQ ID NO: 1, and still more preferably at least 90-95% identical to the nucleic acid sequence of SEQ ID NO: 1 over a region of at least 100 nucleotides. In some instances, the region of percent identity extends over a longer region such as 125, 150, 175, 200, 225 or 250 nucleotides, or over the full length of the encoding region. To identify nucleic acids of the invention, one can employ a nucleotide sequence comparison algorithm such as are known to those of skill in the art. For example, one can use the BLASTN algorithm. Suitable parameters for use in BLASTN are wordlength (W) of 11, M=5 and N=−4.

Alternatively, one can identify a nucleic acid of the invention by hybridizing, under stringent conditions, the nucleic acid of interest to a nucleic acid that includes a polynucleotide sequence of SEQ. ID NOS: 1, 3, 5, and 7. The invention also includes nucleic acids which encode a polypeptide which is immunologically cross reactive with SEQ ID NOS: 2, 4, 6, or 8 subsequences thereof.

Nucleic acid sequences of the present invention can be obtained by methods such as for example, 1) hybridization of genomic or cDNA libraries with probes to detect homologous nucleotide sequences; 2) antibody screening of expression libraries to detect cloned DNA fragments with shared structural features; 3) various amplification procedures such as polymerase chain reaction (PCR) using primers capable of annealing to the nucleic acid of interest; 4) direct chemical synthesis. Suitable primers for amplification of the coding sequence of the genes from cDNA or DNA are depicted in Table 4 below:

| | | |
|---|---|---|
| Gene | Primer | SEQ ID NO |

| | | |
|---|---|---|
| hOST1 | atggagcagcctgtgttcctgatg | 9 |
| | ctagaattcatcatcagagctgagc | 10 |
| hOST2 | atgagtaatgtctcagggatcctgg | 11 |
| | ctacaggtcctccgaggggatcagc | 12 |
| hOST3 | atgccttgcacttgtacctggagg | 13 |
| | tcaggaatccacggatttatctgaag | 14 |
| hOST4 | atggagccgggcaggacccagataa | 15 |
| | ttaggctttgaggttcaagtccagg | 16 |

IV. Production of Transporter Proteins

The transporter proteins of SEQ. ID NOS: 2, 4, 6, and 8 and variants thereof can be produced in prokaryotic or eukaryotic host cells by expression of polynucleotides encoding the transporter proteins. DNA sequences are expressed in hosts after the sequences have been operably linked to an expression control sequence in an expression vector. Expression vectors are typically replicable in the host organisms either as episomes or as an integral part of the host chromosomal DNA. Commonly, expression vectors contain selection markers, e.g., tetracycline resistance or hygromycin resistance, to permit detection and/or selection of those cells transformed with the desired DNA sequences (see, e.g., U.S. Pat. No. 4,704,362).

Typically, the polynucleotide that encodes a polypeptide of the invention is placed under the control of a promoter that is functional in the desired host cell to produce relatively large quantities of a polypeptide of the invention. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the invention provides expression cassettes into which the nucleic acids that encode the polypeptides described herein are incorporated for high level expression in a desired host cell. For expression of the polypeptides in mammalian cells, convenient promoters include CMV promoter (Miller, et al., BioTechniques 7:980), SV40 promoter (de la Luma, et al.,(1998) Gene 62:121), RSV promoter (Yates, et al., Nature 313:812(1985), MMTV promoter (Lee, et al., Nature 294:228(1981)).

Either constitutive or regulated promoters can be used in the present invention. Regulated promoters can be advantageous because the host cells can be grown to high densities before expression of the polypeptides is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, and allow one to control the timing of expression of the polypeptide.

Once expressed, the recombinant polypeptides can be purified, if desired, according to standard procedures of the art, including ammonium sulfate precipitation, affinity columns, ion exchange and/or size exclusivity chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 90 to 95% homogeneity are preferred, and 98 to 99% or more homogeneity are most preferred. Once purified, partially or to homogeneity as desired, the polypeptides may then be used (e.g., treatment of inflammatory diseases in pre-clinical or clinical studies).

To facilitate purification of transporters of the invention, the nucleic acids that encode the polypeptides can also include a coding sequence for an epitope or “tag” for which an affinity binding reagent is available. Examples of suitable epitopes include the myc and V-5 reporter genes; expression vectors useful for recombinant production of polypeptides having these epitopes are commercially available (e.g., Invitrogen (Carlsbad Calif.) vectors pcDNA3.1/Myc-His and pcDNA3.1/V5-His are suitable for expression in mammalian cells; Invitrogen (Carlsbad, Calif.) vectors pBlueBacHis and Gibco (Gaithersburg, Md.) vectors pFastBacHT are suitable for expression in insect cells). Additional expression vectors suitable for attaching a tag to the proteins of the invention, and corresponding detection systems are commercially available (e.g., FLAG” (Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

V. Methods of Identifying Agents, Conjugates or Conjugate Moieties that are Substrates of a Transporter

Agents known or suspected to have pharmacological activity can be screened directly for their capacity to act as substrates of one of the transporters of the invention. Alternatively, conjugate moieties can be screened as substrates, and the conjugate moieties linked to agents having known or suspected pharmacological activity. In such methods, the conjugate moieties can be linked to an agent or other molecule during the screening process. If another molecule is used, the molecule is sometimes chosen to resemble the structure of an agent ultimately intended to be linked to the conjugate moiety for pharmaceutical use. The screening is typically performed on cells expressing a transporter. Optionally, hOst4, or other transporters of the invention, is coexpressed with a co-transporter having the amino acid sequence designated SEQ ID NO:20 (or allelic variants thereof, or variants having at least 90% sequence identity to SEQ ID NO:20 over the entire length of the co-transporter). The co-transporter is preferably encoded by a cDNA sequence designated SEQ ID NO:19 or allelic variants thereof or variants having at least 90% sequence identity thereto over the entire length of the co-transporter. The preceding disclosure with respect to fragments of transporters and nucleic acids encoding the same, and to expression of transporters applies mutatis mutandis to SEQ ID NOS:19 and 20. In some methods, the cells are transfected with DNA encoding a transporter, and optionally a co-transporter. Optionally, hOst4 or other transporter of the invention, is coexpressed with a co-transporter having the amino acid sequence designated SEQ ID NO:20. The activity of some transporters is augmented by a transporter modifying protein. Oocytes, human embryonic kidney cells (HEKs), CaCo-2, MDCK (Madin-Darby canine kidney), CHO (Chinese hamster ovary), Sf9, Sf21, High-5, or any cell line suitable for the expression of transporters may be transfected.

In other methods, natural cells express a transporter. In some methods, a transporter of the invention is the only transporter expressed. In other methods, cells express a transporter of the invention in combination with other transporters, including cotransporters of the exemplified transporters. For example, in some methods, cells expressing at least two of hOst-1 to −4 are used. In still other methods, agents, conjugate moieties or conjugates are screened on different cells expressing different transporters. Agents, conjugate moieties or conjugates can be screened either for specificity for one transporter or for capacity to be substrates to several transporters. In some methods, agents, conjugate moieties or conjugates are screened for capacity to be a substrate for a known transporter that effects transport through an apical plasma membrane of epithelial cells lining the colon, as well as through one of the transporters of the invention. Examples of such transporters are described by W003/065982 (e.g., MCT1, MCT4, ATBO, OCTN2, NADC1 or NADC2). Agents, conjugates, or conjugate moieties that are a substrate for both a transporter expressed in the basolateral membrane of epithelial cells and apical plasma membrane can more easily pass through the epithelial cells lining the intestine. Agents, conjugate moieties or conjugates with specificity for a particular transporter can be useful for limiting uptake to certain tissues or avoiding interaction between drugs. Agents, conjugate moieties or conjugates that are substrates for multiple transporters are useful for maximum uptake. Other types of known transporter for transport of organic solvents include the sodium coupled bile acid transporter, the sodium independent organic anion transporter, the organic anion transporter, and the organic cation transporters (see Kullack et al., Semin. Liver Dis. 20, 273-292 (2000); Borst, J. Natl. Cancer Inst. 92, 1295-1302; Keppler, Semin. Liver Dis. 20, 265-272 (2000); Suzuki, Semin. Liver Dis. 20, 251-263 (2000); Saier, Microbiol. Mol. Biol. Rev. 64 354-411 (2000). Methods of screening agents, conjugates or conjugate moieties for passage through cells bearing a transporter are described in WO 01/20331.

Internalization of a compound evidencing passage through transporters can be detected by detecting a signal from within a cell from any of a variety of reporters. The reporter can be as simple as a label such as a fluorophore, a chromophore, a radioisotope, Confocal imaging can also be used to detect internalization of a label as it provides sufficient spatial resolution to distinguish between fluorescence on a cell surface and fluorescence within a cell; alternatively, confocal imaging can be used to track the movement of compounds over time. In another approach, internalization of a compound is detected using a reporter that is a substrate for an enzyme expressed within a cell. Once the complex is internalized, the substrate is metabolized by the enzyme and generates an optical signal or radioactive decay that is indicative of uptake. Light emission can be monitored by commercial PMT-based instruments or by CCD-based imaging systems. In addition, assay methods utilizing LCMS detection of the transported compounds or electrophysiological signals indicative of transport activity are also employed. Measurements can also be performed in vivo by administering an agent, conjugate or conjugate moiety to an experimental animal and monitoring uptake into the plasma. The agent, conjugate or conjugate moiety can be administered orally or directly into part of the intestine in which a transporter of interest is expressed such as the small intestine or the colon.

In some methods, multiple agents, conjugates or conjugate moieties are screened simultaneously and the identity of each agent, conjugate moiety or conjugate moiety is tracked using tags linked to the agents, conjugates or conjugate moieties. In some methods, a preliminary step is performed to determine binding of an agent, conjugate or conjugate moiety to a transporter. Although not all agents, conjugates or conjugate moieties that bind to a transporter are substrates of the transporter, observation of binding is an indication that allows one to reduce the number of candidate substrates from an initial repertoire. In some methods, substrate capacity of an agent, conjugate or conjugate moiety is tested in comparison with a reference substrate of a transporter. For example, 3H-taurocholate is suitable as a reference. The comparison can either be performed in separate parallel assays in which an agent, conjugate or conjugate moiety under test and the reference substrate are compared for uptake on separate samples of the same cells. Alternatively, the comparison can be performed in a competition format in which an agent, conjugate or conjugate moiety under test and the reference substrate are applied to the same cells. Typically, the agent, conjugate or conjugate moiety and the reference substrate are differentially labeled in such assays.

In such comparative assays, the Vmax of an agent, conjugate moiety, or conjugate comprising an agent and conjugate moiety tested can be compared with that of the reference substrate (e.g., taurocholate or estrone-3-sulfate). If an agent, conjugate moiety or conjugate has a Vmax of at least 1, 5, 10. 20, 50% of the reference substrate for the transporter then the agent, conjugate moiety or conjugate can be considered to be a substrate for the transporter. In general, the higher the Vmax of the agent, conjugate moiety or conjugate relative to that of the reference substrate the better. Therefore, agents, conjugate moieties or conjugates having Vmax's of at least 50%, 100%, 150% or 200% of the Vmax of the reference substrate for the transporter are screened in some methods. The agents to which conjugate moieties are linked can by themselves show little or no detectable substrate activity for the transporter (e.g., Vmax relative to that of a reference substrate of less than 0.1 or 1%).

In some methods, the Vmax of an agent, conjugate moiety or conjugate is also determined relative to the reference substrate for a second transporter. Such screening may reveal that the agent, conjugate moiety or conjugate is a better substrate for one transporter than another. The relative capacities of a substrate for two transporters can be compared by a comparison of the ratios of Vmax of the agent, conjugate moiety or conjugate and taurocholate for the respective transporters.

VI. Agents, Conjugates and Conjugate Moieties to be Screened

Compounds constituting agents, conjugates or conjugate moieties to be screened can be naturally occurring or synthetic molecules. Natural sources include sources such as, e.g., marine microorganisms, algae, plants, and fungi. Alternatively, compounds to be screened can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Compounds can include, e.g., pharmaceuticals, therapeutics, environmental, agricultural, or industrial agents, pollutants, cosmeceuticals, drugs, organic compounds, lipids, glucocorticoids, antibiotics, peptides, sugars, carbohydrates, and chimeric molecules.

Combinatorial libraries can be produced for many types of compounds that can be synthesized in a step-by-step fashion (see e.g., Ellman & Bunin, J Amer Chem Soc, 114:10997, 1992 (benzodiazepine template), WO 95/32184 (oxazolone and aminidine template), WO 95/30642 (dihydrobenzopyran template) and WO 95/35278 (pyrrolidine template). Libraries of compounds are usually synthesized by solid phase chemistry on particle. However, solution-phase library synthesis can also be useful. Strategies for combinatorial synthesis are described by Dolle & Nelson, J. Combinatorial Chemistry 1. 235-282 (1999)) (incorporated by reference in its entirety for all purposes). Synthesis is typically performed in a cyclic fashion with a different monomer or other component being added in each round of synthesis. Some methods are performed by successively fractionating an initial pool. For example, a first round of synthesis is performed on all supports. The supports are then divided into two pools and separate synthesis reactions are performed on each pool. The two pools are then further divided, each into a further two pools and so forth. Other methods employ both splitting and repooling. For example, after an initial round of synthesis, a pool of compounds is split into two for separate syntheses in a second round. Thereafter, aliquots from the separate pools are recombined for a third round of synthesis. Split and pool methods result in a pool of mixed compounds. These methods are particularly amenable for tagging as described in more detail below. The size of libraries generated by such methods can vary from 2 different compounds to 10⁴,10⁶, 10⁸, or 10¹⁰, or any range therebetween.

Preparation of encoded libraries is described in a variety of publications including Needels, et al., Proc. Natl. Acad. Sci. USA 1993, 90, 10700; Ni, et al., J. Med. Chem. 1996, 39, 1601, WO 95/12608, WO 93/06121, WO 94/08051, WO 95/35503 and WO 95/30642 (each of which is incorporated by reference in its entirety for all purposes). Methods for synthesizing encoded libraries typically involve a random combinatorial approach and the chemical and/or enzymatic assembly of monomer units. For example, the method typically includes steps of: (a) apportioning a plurality of solid supports among a plurality of reaction vessels; (b) coupling to the supports in each reaction vessel a first monomer and a first tag using different first monomer and tag combinations in each different reaction vessel; (c) pooling the supports; (d) apportioning the supports among a plurality of reaction vessels; (e) coupling to the first monomer a second monomer and coupling to either the solid support or to the first tag a second tag using different second monomer and second tag combinations in each different reaction vessel; and optionally repeating the coupling and apportioning steps with different tags and different monomers one to twenty or more times. The monomer set can be expanded or contracted from step to step; or the monomer set could be changed completely for the next step (e.g., amino acids in one step, nucleosides in another step, carbohydrates in another step). A monomer unit for peptide synthesis, for example, can include single amino acids or larger peptide units, or both.

Compounds synthesizable by such methods include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Prepared combinatorial libraries are also available from commercial sources (e.g., ChemRx, South San Francisco, Calif.).

Some compounds to be screened are variants of known transporter substrates. Some compounds to be screened are bile salts or acids, steroids, ecosanoids, or natural toxins or analogs thereof, as described by Smith, Am. J. Physiol. 2230, 974-978 (1987); Smith, Am. J. Physiol. 252, G479-G484 (1993); Boyer, Proc. Natl. Acad. Sci. USA 90, 435-438 (1993); Fricker, Biochem. J. 299, 665-670 (1994); Ficker, Biochem J. 299, 665-670 (1994); Ballatori, Am. J. Physiol. 278.

VII. Linkage of Agents to Conjugate Moieties

Conjugates of this invention can be prepared by either by direct conjugation of an agent to a conjugate moiety, wherein the resulting covalent bond is cleavable in vivo, or by covalently coupling a difunctionalized linker precursor with an agent to a conjugate moiety. The linker precursor is selected to contain at least one reactive functionality that is complementary to at least one reactive functionality on the agent and at least one reactive functionality on the conjugate moiety. Such complementary reactive groups are well known in the art as illustrated below:

COMPLEMENTARY BINDING CHEMISTRIES

| First Reactive Group | Second Reactive Group | Linkage |
|---|---|---|
| hydroxyl | carboxylic acid | ester |
| hydroxyl | haloformate | carbonate |
| thiol | carboxylic acid | thioester |
| thiol | haloformate | thiocarbonate |
| amine | carboxylic acid | amide |
| hydroxyl | isocyanate | carbamate |
| hydroxyl | haloformate | carbamate |
| amine | isocyanate | urea |
| carboxylic acid | carboxylic acid | anhydride |
| hydroxyl | phosphorus acid | phosphonate or |
| | | phosphate ester |

In addition to the complementary chemistry of the functional groups on the linker to both the agent and conjugate moiety, the linker (when employed) is also selected to be cleavable in vivo. Cleavable linkers are well known in the art and are selected such that at least one of the covalent bonds of the linker that attaches the agent to the conjugate moiety can be broken in vivo thereby providing for the agent or active metabolite thereof to be available to the systemic blood circulation. The linker is selected such that the reactions required to break the cleavable covalent bond are favored at the physiological site in vivo which permits agent (or active metabolite thereof) release into the systemic blood circulation. The selection of suitable cleavable linkers to provide effective concentrations of the agent or active metabolite thereof for release into the systemic blood circulation can be evaluated using endogenous enzymes in standard in vitro assays to provide a correlation to in vivo cleavage of the agent or active metabolite thereof from the conjugate, as is well known in the art. It is recognized that the exact cleavage mechanism employed is not critical to the methods of this invention provided, of course, that the conjugate cleaves in vivo in some form to provide for the agent or active metabolite thereof for sustained release into the systemic blood circulation.

In another approach, a conjugate moiety and agent are each attached to moieties having mutual affinity for each (e.g., avidin or streptavidin and biotin, or hexahistidine and Ni²⁺). In another approach, both agent and conjugate moiety are linked to a solid phase. Examples of such supports include nanoparticles (see, e.g., U.S. Pat. Nos. 5,578,325 and 5,543,158), molecular scaffolds, liposomes (see, e.g., Deshmuck, D. S., et al., Life Sci. 28:239-242 (1990), and Aramaki, Y., et al., Pharm. Res. 10:1228-1231 (1993), protein cochleates (stable protein-phospholipid-calcium precipitates; see, e.g., Chen et al., J. Contr. Rel. 42:263-272 (1996), and clathrate complexes. These supports can be used to attach other active molecules. Certain supports such as nanoparticles can also be used to encapsulate desired compounds. An agent can be linked to a support via a cleavable linkage allowing separation of the agent after uptake through a transporter.

Examples of cleavable linkers suitable for use as described above include nucleic acids with one or more restriction sites, or peptides with protease cleavage sites (see, e.g., U.S. Pat. No. 5,382,513). Other exemplary linkers that can be used are available from Pierce Chemical Company in Rockford, Ill.; suitable linkers are also described in EPA 188,256; U.S. Pat. Nos. 4,671,958; 4,659,839; 4,414,148; 4,669,784; 4,680,338, 4,569,789 and 4,589,071; and in Eggenweiler, H. M, Drug Discovery Today, 3: 552 (1998), each of which is incorporated in its entirety for all purposes.

There are many existing drugs for which uptake can be improved through the colon. Drugs suitable for conversion to prodrugs that are capable of uptake from the colon typically contain one or more of the following functional groups to which a promoiety may be conjugated: primary or secondary amino groups, hydroxyl groups, carboxylic acid groups, phosphonic acid groups, or phosphoric acid groups.

Examples of drugs containing carboxyl groups include, for instance, angiotensin-converting enzyme inhibitors such as alecapril, captopril, 1-[4-carboxy-2-methyl-2R,4R-pentanoyl]2,3-dihydro-2S-indole-2-carboxylic acid, enalaprilic acid, lisinopril, N-cyclopentyl-N-[3-[(2,2-dimethyl-l-oxopropyl)thio]-2-methyl-1-oxopropyl]glycine, pivopril, quinaprilat, (2R, 4R)-2-hydroxyphenyl)-3-(3-mercaptopropionyl)-4-thiazolidinecarboxylic acid, (S) benzamido-4-oxo-6-phenylhexenoyl-2-carboxypyrrolidine, [2S-1 [R*(R*))]] 2α, 3αβ, 7αβ]-1 [2-[[1-carboxy-3-phenylpropyl]-amino]-1 -oxopropyl]octahydro-1H-indole-carbolic acid, [3S-1[R*(R*))]], 3R*]-2-[2-[[1-carboxy-3-phenylpropyl]-amino]-1-oxopropyl]-1,2,3,4-tetrahydro-3-isoquinolone carboxylic acid, and tiopronin; cephalosporin antibiotics such as cefaclor, cefadroxil, cefamandole, cefatrizine, cefazedone, cefazuflur, cefazolin, cefbuperazone, cefixime, cefmenoxime, cefmetazole, cefodizime, cefonicid, cefoperazone, ceforanide, cefotaxime, cefotefan, cefotiam, cefoxitin, cefpimizole, cefpirome, cefpodoxime, cefroxadine, cefsulodin, cefpiramide, ceftazidime, ceftezole, ceftizoxime, ceftriaxone, cefuroxime, cephacetrile, cephalexin, cephaloglycin, cephaloridine, cephalosporin, cephanone, cephradine, and latamoxef; penicillins such as amoxycillin, ampicillin, apalcillin, azidocillin, azlocillin, benzylpencillin, carbenicillin, carfecillin, carindacillin, cloxacillin, cyclacillin, dicloxacillin, epicillin, flucloxacillin, hetacillin, methicillin, mezlocillin, nafcillin, oxacillin, phenethicillin, piperazillin, sulbenicllin, temocillin, and ticarcillin; thrombin inhibitors such as argatroban, melagatran, and napsagatran; influenza neuraminidase inhibitors such as zanamivir and peramivir; non-steroidal antiinflammatory agents such as acametacin, alclofenac, alminoprofen, aspirin (acetylsalicylic acid), 4-biphenylacetic acid, bucloxic acid, carprofen, cinchofen, cinmetacin, clometacin, clonixin, diclenofac, diflunisal, etodolac, fenbufen, fenclofenac, fenclosic acid, fenoprofen, ferobufen, flufenamic acid, flufenisal, flurbiprofin, fluprofen, flutiazin, ibufenac, ibuprofen, indomethacin, indoprofen, ketoprofen, ketorolac, lonazolac, loxoprofen, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, pirprofen, prodolic acid, salicylic acid, salicylsalicylic acid, sulindac, suprofen, tiaprofenic acid, tolfenamic acid, tolmetin and zopemirac; prostaglandins such as ciprostene, 16-deoxy-16-hydroxy-16-vinyl prostaglandin E₂,6,16-dimethylprostaglandin E₂, epoprostostenol, meteneprost, nileprost, prostacyclin, prostaglandins E₁, E₂, or F_{2α,}and thromboxane A₂; quinolone antibiotics such as acrosoxacin, cinoxacin, ciprofloxacin, enoxacin, flumequine, naladixic acid, norfloxacin, ofloxacin, oxolinic acid, pefloxacin, pipemidic acid, and piromidic acid; other antibiotics such as aztreonam, imipenem, meropenem, and related carbopenem antibiotics.

Representative drugs containing amine groups include: acebutalol, albuterol, alprenolol, atenolol, bunolol, bupropion, butopamine, butoxamine, carbuterol, cartelolol, colterol, deterenol, dexpropanolol, diacetolol, dobutamine, exaprolol, exprenolol, fenoterol, fenyripol, labotolol, levobunolol, metolol, metaproterenol, metoprolol, nadolol, pamatolol, penbutalol, pindolol, pirbuterol, practolol, prenalterol, primidolol, prizidilol, procaterol, propanolol, quinterenol, rimiterol, ritodrine, solotol, soterenol, sulfiniolol, sulfinterol, sulictidil, tazaolol, terbutaline, timolol, tiprenolol, tipridil, tolamolol, thiabendazole, albendazole, albutoin, alendronate, alinidine, alizapride, amiloride, aminorex, aprinocid, cambendazole, cimetidine, cisapride, clonidine, cyclobenzadole, delavirdine, efegatrin, etintidine, fenbendazole, fenmetazole, flubendazole, fludorex, gabapentin, icadronate, lobendazole, mebendazole, metazoline, metoclopramide, methylphenidate, mexiletine, neridronate, nocodazole, oxfendazole, oxibendazole, oxmetidine, pamidronate, parbendazole, pramipexole, prazosin, pregabalin, procainamide, ranitidine, tetrahydrazoline, tiamenidine, tinazoline, tiotidine, tocainide, tolazoline, tramazoline, xylometazoline, dimethoxyphenethylamine, N-[3(R)-[2-piperidin-4-yl)ethyl]-2-piperidone-1-yl]acetyl-3(R)-methyl-β-alanine, adrenolone, aletamine, amidephrine, amphetamine, aspartame, bamethan, betahistine, carbidopa, clorprenaline, chlortermine, dopamine, L-Dopa, ephrinephrine etryptamine, fenfluramine, methyldopamine, norepinephrine, tocainide, enviroxime, nifedipine, nimodipine, triamterene, norfloxacin, and similar compounds such as pipedemic acid, 1-ethyl-6-fluoro-1,4dihydro-4-oxo-7-(1-piperazinyl)-1,8-napthyridine-3-carboxylic acid, 1-cyclopropyl-6-fluoro-1, and 4-dihydro-4-oxo-7-(piperazinyl)-3-quinolinecarboxylic acid.

Representative drugs containing hydroxy groups include: steroidal hormones such as allylestrenol, cingestol, dehydroepiandrosteron, dienostrol, diethylstilbestrol, dimethisteron, ethyneron, ethynodiol, estradiol, estron, ethinyl estradiol, ethisteron, lynestrenol, mestranol, methyl testosterone, norethindron, norgestrel, norvinsteron, oxogeston, quinestrol, testosterone, and tigestol; tranquilizers such as dofexazepam, hydroxyzin, lorazepam, and oxazepam; neuroleptics such as acetophenazine, carphenazine, fluphenazine, perphenyzine, and piperaetazine; cytostatics such as aclarubicin, cytarabine, decitabine, daunorubicin, dihydro-5-azacytidine, doxorubicin, epirubicin, estramustin, etoposide, fludarabine, gemcitabine, 7-hydroxychlorpromazin, nelarabine, neplanocin A, pentostatin, podophyllotoxin, tezacitabine, troxacitabine, vinblastin, vincristin, and vindesin; hormones and hormone antagonists such as buserilin, gonadoliberin, icatibrant, and leuprorelin acetate; antihistamines such as terphenadine; analgesics such as diflunisal, naproxol, paracetamol, salicylamide, and salicyclic acid; antibiotics such as azidamphenicol, azithromycin, camptothecin, cefamandol, chloramphenicol, clarithromycin, clavulanic acid, clindamycin, demeclocyclin, doxycyclin, erythromycin, gentamycin, imipenem, latamoxef, metronidazole, neomycin, novobiocin, oleandomycin, oxytetracyclin, tetracycline, thiamenicol, and tobramycin; antivirals such as acyclovir, dideoxydidehydrocytidine, dideoxycytosine, 1-(2-deoxy-2-methylene-beta-D-erythro-pentofuranosyl)cytidine, fluoro-dideoxydidehydrocytidine, fluorodideoxycytosine, FMAU (1-(2-deoxy-2-fluoro-beta-D-arabinofuranosyl)thymine), deoxy-5-fluoro-3′-thiacytidine, 2′-fluoro-ara-dideoxyinosine, ganciclovir, lamivudine, penciclovir, SddC, stavudine, 5-trifluoromethyl-2′-deoxyuridine, zalcitabine, and zidovudine; bisphosphonates such as EB-1053 (1-hydroxy-3-(1-pyrrolidinyl)propylidene-1,1-bisphosphonate), etidronate, ibandronate, olpadronate, residronate, 1-hydroxy-2-(imidazo [1,2-a] pyridin-3-yl) ethylidene]-bisphosphonic acid, and zolendronate; protease inhibitors such as ciprokiren, enalkiren, ritonavir, saquinavir, and terlakiren; prostaglandins such as arbaprostil, carboprost, misoprostil, and prostacydin; antidepressives such as 8-hydroxychlorimipramine and 2-hydroxyimipramine; antihypertonics such as sotarol and fenoldopam; anticholinerogenics such as biperidine, procyclidin and trihexyphenidal; antiallergenics such as cromolyn; glucocorticoids such as betamethasone, budenosid, chlorprednison, clobetasol, clobetasone, corticosteron, cortisone, cortodexon, dexamethason, flucortolon, fludrocortisone, flumethasone, flunisolid, fluprednisolon, flurandrenolide, flurandrenolon acetonide, hydrocortisone, meprednisone, methylpresnisolon, paramethasone, prednisolon, prednisol, triamcinolon, and triamcinolon acetonide; narcotic agonists and antagonists such as apomorphine, buprenorphine, butorphanol, codein, cyclazocin, hydromorphon, ketobemidon, levallorphan, levorphanol, metazocin, morphine, nalbuphin, nalmefen, naloxon, nalorphine, naltrexon, oxycodon, oxymorphon, and pentazocin; stimulants such asmazindol and pseudoephidrine; anaesthetics such as hydroxydion and propofol; p-receptor blockers such as acebutolol, albuterol, alprenolol, atenolol, betazolol, bucindolol, cartelolol, celiprolol, cetamolol, labetalol, levobunelol, metoprolol, metipranolol, nadolol, oxyprenolol, pindolol, propanolol, and timolol; α-sympathomimetics such as adrenalin, metaraminol, midodrin, norfenefrin, octapamine, oxedrin, oxilofrin, oximetazolin, and phenylefrin; β-sympathomimetics such as bamethan, clenbuterol, fenoterol, hexoprenalin, isoprenalin, isoxsuprin, orciprenalin, reproterol, salbutamol, and terbutalin; bronchodilators such as carbuterol, dyphillin, etophyllin, fenoterol, pirbuterol, rimiterol and terbutalin; cardiotonics such as digitoxin, dobutamin, etilefrin, and prenalterol; antimycotics such as amphotericin B, chlorphenesin, nystatin, and perimycin; anticoagulants such as acenocoumarol, dicoumarol, phenprocoumon, and warfarin; vasodilators such as bamethan, dipyrimadol, diprophyllin, isoxsuprin, vincamin and xantinol nicotinate; antihypocholesteremics such as compactin, eptastatin, mevinolin, and simvastatin; miscellaneous drugs such as bromperidol (antipsychotic), dithranol (psoriasis) ergotamine (migraine) ivermectin (antihelminthic), metronidazole and secnizadole (antiprotozoals), nandrolon (anabolic), propafenon and quinadine (antiarythmics), quetiapine (CNS), serotonin (neurotransmitter), and silybin (hepatic disturbance).

Representative drugs containing phosphonic acid moieties include: adefovir, alendronate, (N6-[2-methylthio)ethyl]-2-[3,3,3-trifluoropropylthio]-5′-adenylic acid, BMS-187745 (a squalene synthase inhibitor from Bristol-Meyers Squibb Inc.), ceronapril, CGP-24592 (Novartis, Inc.), DL-(E)-2-amino-4-methyl-5-phosphono-3-pentenoic acid; 4-methyl-APPA, CGP-39551 (ethyl esters of (DL-[E]-2-amino-4-methyl-5-phosphono-3-pentenoic acid)), CGP-40116 (a competitive NMDA antagonist by Novartis Inc.), cidofovir, clodronate, EB-1053 (1-hydroxy-3-(1-pyrrolidinyl)propylidene-1,1-bisphosphonate), etidronate, fanapanel, foscamet, fosfomycin, fosinopril, fosinoprilat, ibandronate, midafotel, neridronate, olpadronate, pamidronate, residronate, tenofovir, tiludronate, [2-(8,9-dioxo-2,6-diazabicyclo[5.2.0]non-1(7)-en-2-yl)ethyl]phosphonic acid, 1-hydroxy-2-(imidazo [1,2-a] pyridin-3-yl) ethylidene]-bisphosphonic acid, and zolendronate.

Representative drugs containing phosphoric acid moieties include: bucladesine, choline alfoscerate, citocoline, fludarabine phosphate, fosopamine, GP-668, perifosine, triciribine phosphate, and phosphate derivatives of nucleoside analogs which require phophorylation for activity, such as lamivudine, acyclovir, azidothymidine, E-5-(2-bromovinyl)-2′-deoxyuridine, dideoxycytosine, dideoxyinosine, FMAU (1-(2-deoxy-2-fluoro-beta-D-arabinofuranosyl)thymine), deoxy-5-fluoro-3′-thiacytidine, ganciclovir, gemcitabine, (R)-9-[4-Hydroxy-2-(hydroxymethy)butyl]guanine, lamivudine, penciclovir and the like.

Preferred drugs for modification to prodrugs capable of intestional absorption and incorporation -into sustained release formulations include the following compounds: analgesics and/or antiinflammatory agents selected from the group consisting of acetaminophen, buprenorphine, diclofenac, diflunisal, fenoprofen, ibuprofen, indomethacin, ketoprofen, mefenamic acid, meptazinol, morphine, oxycodone, pentazocine, pethidine, tolmetin, and tramadol; antihypertensive agents selected from the group consisting of captopril, diltiazem, methyldopa, metoprolol, prazosin, propranolol, quinapril, sotalol, and timolol; antibiotic agents selected from the group consisting of amoxicillin, ampicillin, aztreonam, cefaclor, cefadroxil, cefixime, cefotaxime, cefoxitin, cefpodoxime, ceftizoxime, ceftriaxone, cefuroxime, cephalexin, ciproflaxacin, clindamycin, erythromycin, imipenem, mandol, meropenem, metronidazole, and tobramycin; antiviral agents selected from the group consisting of acyclovir, delavirdine, didanosine, foscarnet, ganciclovir, indinavir, lamivudine, nelfinavir, penciclovir, ritonavir, saquinavir, stavudine, zalcitabine, and zidovudine; bronchodilator and or anti-asthmatic agents selected from the group consisting of salbutamol and terbutaline; antiarrhythmic agents selected from the group consisting of mexiletine, procainamide, and tocainide; centrally acting substances selected from the group consisting of baclofen, benserazide, bupropion, carbidopa, gabapentin, levodopa, methylphenildate, pramipexole, pregabalin, quetiapine, ropinirole, and vigabatrin; cytostatics and metastasis inhibitors selected from the group consisting of cytarabine, decitabine, docetaxal, flutamide, gemcitabine, paclitaxel, and pentostatin; and, agents for treatment of gastrointestinal disorders selected from the group consisting of cisapride, metoclopramide, and misoprostol.

VIII. Pharmaceutical Compositions and Methods of Treatment

Agents that are themselves substrates for a transporter or which are linked to conjugate moieties that are substrates for a transporter can be can be incorporated into pharmaceutical compositions. Usually, although not necessarily, such pharmaceutical compositions are designed for oral administration. Oral administration of such compositions results in uptake through the intestine via a transporter and entry into the systemic circulation. The pharmaceutical composition can thus be efficiently delivered to a wide range of tissues in the body.

Agents optionally linked to a conjugate moiety are combined with pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can also include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like (see, e.g., Remington's Pharmaceutical Sciences, Mace Publishing Company, Philadelphia, Pa., 17th ed. (1985); for a brief review of methods for drug delivery, see, Langer, Science 249:1527-1533 (1990); each of these references is incorporated by reference in its entirety).

Pharmaceutical compositions for oral administration can be in the form of e.g., tablets, pills, powders, lozenges, sachets, cachets, elixirs, suspensions, emulsions, solutions, or syrups. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. Preserving agents such as methyl-and propylhydroxy-benzoates; sweetening agents; and flavoring agents can also be included. Depending on the formulation, compositions can provide quick, sustained or delayed release of the active ingredient after administration to the patient. The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 mg to about 2 g of the active agent.

The compositions can be administered for prophylactic and/or therapeutic treatments. A therapeutic amount is an amount sufficient to remedy a disease state or symptoms, or otherwise prevent, hinder, retard, or reverse the progression of disease or any other undesirable symptoms in any way whatsoever. In prophylactic applications, compositions are administered to a patient susceptible to or otherwise at risk of a particular disease or infection. Hence, a “prophylactically effective” is an amount sufficient to prevent, hinder or retard a disease state or its symptoms. In either instance, the precise amount of compound contained in the composition depends on the patient's state of health and weight.

An appropriate dosage of the pharmaceutical composition is readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

The pharmaceutical compositions can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods. The route of administration depends in part on the chemical composition of the active compound and any carriers.

The components of pharmaceutical compositions are preferably of high purity and are substantially free of potentially harmful contaminants (e.g., at least National Food (NF) grade, generally at least analytical grade, and more typically at least pharmaceutical grade). To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions. Compositions for oral administration need not be sterile or substantially isotonic but are usually made under GMP conditions.

IX. Other Applications of Transporters

1. Antibodies

The transporters of the invention can be used to generate antibodies. The antibodies can be polyclonal antibodies, distinct monoclonal antibodies or pooled monoclonal antibodies with different epitopic specificities. Monoclonal antibodies are made from antigen-containing fragments of the protein by standard procedures according to the type of antibody (see, e.g., Kohler, et al., Nature, 256:495, (1975); and Harlow & Lane, Antibodies, A Laboratory Manual (C.S.H.P., NY, 1988) Queen et al., Proc. Natl. Acad. Sci. USA 86:10029-10033 (1989) and WO 90/07861; Dower et al., WO 91/17271 and McCafferty et al., WO 92/01047 (each of which is incorporated by reference for all purposes). Nonhuman antibodies are typically made by immunizing a nonhuman animal, such as a mouse, harvesting B-cells from the animal, immortalizing the cells to produce hybridomas, and selecting a hybridoma secreting an antibody having the desired binding characterisitics of a tranporter. The antibodies of the invention can be chimeric, humanized, human, mouse or other species. Phage display technology can also be used to mutagenize CDR regions of antibodies previously shown to have affinity for the peptides of the present invention. Preferably such antibodies do not specifically bind to the skate Ost transporter. The antibodies can be further purified, for example, by binding to and elution from a support to which the polypeptide or a peptide to which the antibodies were raised is bound.

Antibodies of the invention are useful, for example, in screening cDNA expression libraries and for identifying clones containing cDNA inserts which encode structurally-related, immunocrossreactive proteins. See, for example, Aruffo & Seed, Proc. Natl. Acad. Sci. USA 84:8573-8577 (1977) (incorporated herein by reference in its entirety for all purposes). Antibodies are also useful to identify and/or purify immunocrossreactive proteins that are structurally related to SEQ ID NOS: 2, 4, 6, or 8 to fragments thereof used to generate the antibody.

2. Expression Monitoring Arrays

Nucleic acids encoding the transporters of the invention are also useful for inclusion on a GeneChip™ array or the like for use in expression monitoring (see U.S. Pat. No. 6,040,138,. EP 853, 679 and W097/27317). Such arrays typically contain oligonucleotide or cDNA probes to allow detection of large numbers of mRNAs within a mixture. Many of the nucleic acids included in such arrays are from genes or ESTs that have not been well characterized. Such arrays are often used to compare expression profiles between different tissues or between different conditions of the same tissue (healthy vs. diseased or drug-treated vs. control) to identify differentially expressed transcripts. The differentially expressed transcripts are then useful e.g., for diagnosis of disease states, or to characterize responses of drugs. The nucleic acids of the invention can be included on GeneChip™ arrays or the like together with probes containing a variety of other genes. The present nucleic acids are particularly useful for inclusion in GeneChip™ arrays for analyzing the transport capacity of a cell and effects of drugs on the same. Nucleic acids encoding the transporters of the invention can be combined with nucleic acids encoding other transporters molecules. 3. Diagnosis

cDNA or genomic DNA of hOst-1, -2, -3 and 4, can be sequenced and compared with the exemplary sequences of SEQ. ID NOS: 1, 3, 5, and 7. Variation from one of these sequences, particularly a nucleic acid substitution giving rise to a nonconservative amino acid substitution can be indicative of disease. To perform such analysis, the presence or absence of one or more polymorphic forms (i.e., a polymorphic set) is determined for a set of the individuals, some of whom exhibit a particular trait, and some of which exhibit lack of the trait. The alleles of each polymorphism of the set are then reviewed to determine whether the presence or absence of a particular allele is associated with the trait of interest. Correlation can be performed by standard statistical methods such as a chi-squared test and statistically significant correlations between polymorphic form(s) and phenotypic characteristics are noted. Polymorphisms found to correlate with the disease trait can then be used as a basis for a genetic trait.

4. Screening for agents that agonize or antagonize transporter function

The transporters of the invention are useful for screening for agents that agonize or antagonize transporter function. Such agents can be useful as drugs in compensating for genetic variations affecting transporter functions. For example, an agonist of transporter function is useful in a patient having a genetic variation that decreases endogenous transport function, and an antagonist is useful in a patient having a genetic variation that increases endogenous transport function. An agonist is also useful in patients having normal or defective transporter function to increase uptake of another drug against a different target. The transporters can also be used to screen known drugs against targets other than transporters to determine whether the drug has an incidental effect on transport activity. A drug that has an incidental agonist activity should generally be avoided in patients having atypically high levels of transporter activity, and an antagonist should generally be avoided in patients having an atypically low level of endogenous transport activity.

Agents can be screened in cells transfected with nucleic acids encoding a transporter of the invention or in transgenic animals expressing a transporter of the invention as a transgene. Activity of an agent is monitored from its effect on transport of a known substrate such as taurocholate. Agents for screening can be obtained by producing and screening large combinatorial libraries, as described above, or can be known drugs.

Although the foregoing invention has been described in some detail for purposes of clarity and understanding, it will be clear to one skilled in the art from a reading of this disclosure that various changes in form and detail can be made without departing from the true scope of the invention. The above examples are provided to illustrate the invention, but not to limit its scope; other variants of the invention will be readily apparent to those of ordinary skill in the and are encompassed by the claims of the invention. The scope of the invention should, therefore, be determined not with reference to the above description, but instead should be determined with reference to the appended claims along with their full scope of equivalents. All publications, references, and patent documents cited in this application are incorporated by reference in their entirety for all purposes to the same extent as if each individual publication or patent document were so individually denoted.

mRNA Abundance, transcripts/reaction, normalized to GAPDH

| | Human Intestine Mucosal Biopsy Samples | | | | | | | | | | | |
|---|---|---|---|---|---|---|---|---|---|---|---|---|
| | | DU- | | | | T. | S. | | | | | |
| | STOMACH | ODENUM | ILEUM | CECUM | A. COLON | COLON | COLON | RECTUM | KIDNEY | LIVER | OVARY | TESTIS |
| OST-1 | 15218 | 16789 | 12214 | 9644 | 7730 | 9050 | 10163 | 7957 | 18188 | 9743 | 12498 | 2367 |
| OST-2 | 10033 | 17643 | 15518 | 6728 | 10112 | 11130 | 10712 | 6604 | 1600 | 4987 | 0 | 750 |
| OST-3 | 1530 | 6970 | 9449 | 8378 | 8881 | 9471 | 10060 | 1639 | 1654 | 2993 | N.D. | N.D. |
| OST4 | 4866 | 42267 | 65347 | 24009 | 51725 | 55623 | 19137 | 1242 | 11058 | 55863 | 9937 | 24683 |
| (OST- | | | | | | | | | | | | |
| alpha) | | | | | | | | | | | | |

What is claimed is:

1. An isolated polypeptide having an amino acid sequence at least 80% identical to an amino acid sequence as set forth in a sequence selected from the group consisting of SEQ ID NOS: 2, 4, 6, and 8 over a region at least 40 amino acids in length when compared using the BLASTP algorithm with a wordlength (W) of 3, and the BLOSUM62 scoring matrix.

2. The isolated polypeptide of claim 1 selected from the group consisting of SEQ ID NOS: 2,4,6, and 8.

3. The isolated polypeptide of claim 1 that specifically binds to an antibody that specifically binds to a polypeptide selected from the group consisting of SEQ. ID NOS: 2, 4, 6, and8.

4. An isolated nucleic acid having a sequence that is at least 80% identical to a polynucleotide having a sequence selected from the group consisting of SEQ ID NO: 1, 3, 5, and 7 over a region of at least 100 nucleotides in length when compared using the BLASTN algorithm with a wordlength (W) of 11, M=5, and N=−4.

5. The isolated nucleic acid of claim 4 that hybridizes to a sequence selected from the group consisting of SEQ ID NOS: 1, 3, 5, and 7 under conditions of high stringency including 50% formamide, 5×SSC, 5×Denhardt's solution, 10 mM sodium phosphate, pH 6.5, 100 μg/ml salmon sperm DNA and at 420° C.

6. The isolated nucleic acid of claim 1 having a sequence selected from the group consisting of SEQ. ID NOS: 1, 3, 5, and 7.

7. A vector comprising the isolated nucleic acid of claim 4 operably linked to a heterologous promoter.

8. A method of screening whether an agent, conjugate or conjugate moiety is a substrate of a transporter, comprising:
providing a cell expressing a nucleic acid as defined by claim 4 to produce a transporter encoded by the nucleic acid in an outermembrane of the cell;
contacting the cell with an agent, conjugate moiety or conjugate; and
determining whether the agent, conjugate moiety or conjugate passes through the transporter.

9. The method of claim 7, wherein transporter encoded by the nucleic acid has the sequence of SEQ. ID NO: 2.

10. The method of claim 9, wherein the cell is a Chinese hamster ovary cell, a human embryonic kidney cell or an oocyte.

11. A method of screening whether an agent, conjugate or conjugate moiety binds to a transporter;
contacting a transporter having a sequence as defined in claim 1 with an agent, conjugate or conjugate moiety;
detecting presence or absence of binding between the agent, conjugate or conjugate moiety and the transporter.

12. The method of claim 11, wherein transporter encoded by the nucleic acid has the sequence of SEQ. ID NO: 2.

13. A conjugate comprising an agent linked to a conjugate moiety for a transporter having an amino acid sequence as defined by claim 1, wherein the conjugate shows a Vmax of at least 1% of taurocholate for the transporter wherein the agent has a pharmaceutical activity without the conjugate moiety, and the conjugate has a greater Vmax for the transporter than the agent without the conjugate moiety.

14. A method of manufacturing a pharmaceutical composition, comprising;
linking an agent to a conjugate moiety to form a conjugate wherein the conjugate is transported by a transporter as defined by claim 1 with a higher Vmax than the agent alone;
formulating the conjugate with a carrier as a pharmaceutical composition.

15. A method of treatment comprising;
administering to a patient a conjugate comprising an agent linked to a conjugate moiety wherein the conjugate is transported by a transporter as defined by claim 1 with a higher Vmax than the agent alone.

16. The method of claim 12, wherein the conjugate is administered orally to the patient.

17. The method of claim 12, wherein the conjugate is administered intravenously to the patient.

18. An antibody that specifically binds to a polypeptide having an amino acid sequence designated SEQ ID NO:2, 4, 6 or 8.

19. The antibody of claim 18 that is a monoclonal antibody.

20. The antibody of claim 18 that is selected from the group consisting of a mouse antibody, a chimeric antibody, a humanized antibody or a human antibody.

21. A method of producing an antibody, comprising
immunizing a mammal with a protein comprising an amino acid sequence selected from the group consisting of SEQ ID NO:2, 4, 6 and 8, and immunenic fragments thereof;
collecting B cells from the mammal;
immortalizing the B-cells
selecting an immortalized cells producing an antibody that specifically binds to a protein comprising an aimino acid sequence selected from the group consisting of SEQ ID NO:2, 4, 6 and 8.

22. A method of screening agents, conjugates or conjugate moieties for capacity to be substrates for a transporter, comprsing
providing a cell expressing a transporter comprising an amino acid sequence selected from the group consisting of SEQ ID NOS: 2, 4, 6 and 8, the transporter being situated in the plasma membrane of the cell;
contacting the cell with an agent, conjugate or conjugate moiety; and
determining whether the agent, conjugate or conjugate moiety passes through the plasma membrane via the transporter.

23. A method of screening agents, conjugates or conjugate moieties for capacity to agonize or antagonize a transporter, comprising contacting a cell expressing a transporter comprising an amino acid sequence selected from the group consisting of SEQ ID NOS: 2, 4, 6 and 8, the transporter being situated in the plasma membrane of the cell; with an agent, conjugate or conjugate moiety and a known substrate of the transporter;
determining whether the agent agonizes or antagonizes uptake of the known substrate into the cell in comparison with a control cell expressing the transporter contacted with known substrate without the agent, conjugate or conjugate moiety.

24. The method of claim 23, wherein the known substrate is taurocholate or estrone-3-sulfate.

25. An isolated nucleic acid having a sequence that is at least 80% identical to a polynucleotide having a sequence of SEQ ID NO: 19 over a region of at least 100 nucleotides in length when compared using the BLASTN algorithm with a wordlength (W) of 11, M=5, and N=−4.

26. The isolated nucleic acid of claim 25 that hybridizes to a polynucleotide having a sequence of SEQ ID NO: 19 under conditions of high stringency including 50% formamide, 5×SSC, 5×Denhardt's solution, 10 mM sodium phosphate, pH 6.5, 100 μg/ml salmon sperm DNA and at 42° C.

27. The isolated nucleic acid of claim 25 having a sequence of SEQ. ID NOS: 19.
